(19) United States
(12) Patent Application Publication
(10) Pub. No.: US 2025/0022549 A1
(43) Pub. Date: Jan. 16, 2025

(54) INTERVENTION SYSTEM AND METHOD

(71) Applicant: BRAINPATCH LTD, Hayes (GB)

(72) Inventors: Nickolai VYSOKOV, London Kent (GB); Yaroslav GACHSHENKO, Almaty (KZ); Anna TARASENKO, London (GB)

(73) Assignee: BRAINPATCH LTD, Hayes (GB)

(21) Appl. No.: 18/274,782

(22) PCT Filed: Jan. 28, 2022
(86) PCT No.: PCT/IB2022/050765

§ 371 Date: Jul. 28, 2023

(30) Foreign Application Priority Data
Jan. 29, 2021 (GB) 2101229.9

(51) Int. Cl. G16H 10/20

(52) U.S. Cl. CPC G16H 10/20

(57) ABSTRACT

There is disclosed an intervention system that, automatically performs unbiased testing of at least one subject under investigative or experimental study, comprising an input device for collecting bibliographic and physiological information from the at least one subject; a memory module for storing the collected physiological information; an arrangement for providing at least two different types of interventions to the at least one subject; and a data processing arrangement that operates the stimulation arrangement for a random group of subjects, wherein the random group of subjects is a subset of the at least one subject, generates randomization files corresponding to the random group of subjects, and stores the randomization files in the memory module, associates the collected physiological information with the randomization files to generate associated information; and performs at least one intervention to the at least one subject, based on the associated information, characterized in that the randomization files are generated using at least one randomization algorithm.

### TECHNICAL FIELD

The present disclosure relates to an automated system delivering interventions and methods of using such automated systems. More particularly, the present disclosure relates to an intervention system that automatically performs unbiased randomization of subjects for investigative or experimental study, research purposes and home testing.

### BACKGROUND

Conventional experimental or investigative studies (such as clinical trials and research interventions) are expensive and involve either inpatient trials or frequent visits of the subjects to hospital or research facility. Also, the conventional studies for clinical applications involve many other people including doctors and nursing staff of the hospital delivering the treatments. The existing golden standard in study design and execution is a process called “double-blind study”, where neither the patient nor the doctor know whether they are delivering the experimental treatment or a control treatment. Some low-risk treatments could be delivered at home, but where it is necessary to blind the patient for a study, such trial still requires the patient's visit to the hospital facility. The latter has become a problem particularly exacerbated in the context of the global pandemic and highlights a clear need for novel inventive approaches of testing and blinding.

The requirement for regular appearance of participants at a monitoring facility has a number of disadvantages, particularly for trials of interventional drugs. These disadvantages include the burden on the researchers and/or their assistants to spend time scheduling visits, to conduct interviews with each participant, and to compile data on the effectiveness of each substance undergoing test. This burden is magnified by the number of substances and placebos involved in the trial. The need for a large number of participants for statistical accuracy further increases the burden, both in terms of time and resources. Conventional trials having many participants also usually involve multiple test facilities, and staff and resources at each facility in order to conduct the trial there.

Currently, a company organizing clinical trials needs to recruit in-house specialists who design a trial and doctors at the hospital, and in most countries to reimburse them for their time in performing the trial and for working with the patients. The specialists need to design a protocol that would importantly blind the doctor and the patient to the intervention type and ensure, through questionnaires or other means, that they are indeed blinded. Normally the in-house specialist, who is not directly involved with the patients, is not blinded and there is always a chance of inadvertently or maliciously passing the information.

In some interventions, particularly within the industry of non-invasive brain stimulation, the placebo effect and controlling for it has been a critical problem and proper blinding is the biggest cost and a common source of error in the trials.

There are many existing study design(s), for example, a parallel design where different people receive different interventions or a cross-over design where the same person/patient receives both placebo and real treatment in a random sequence. Also, various software packages are used to generate randomisation protocols. However, whenever there is a piece of medical equipment involved that is not factory pre-set to work either as a control or as the real intervention, the clinician in contact with the patient has to operate the medical equipment to deliver one or the other and is thus not blinded.

To circumvent this there exist some systems that use pre-set codes pre-determined in the equipment to deliver either control or real intervention to ensure blinding of the patient and the doctor. Such systems however still involve a clinician who is given the code and then passes it to the patient, as there is no direct communication between the device and the cloud. So, such devices are not really able to automate the blinding process nor implement adaptive trial design. Moreover, the clinician is required to collect the data from the patient and monitor the subjects' well-being and as the device is not connected to the cloud, it is the only means of communicating between the device and the trial organiser. Once such devices have been used for a particular trial, or if they need to be re-purposed on the fly halfway through the trial (e.g. for safety reasons), they need to be returned to the manufacturer for re-purposing.

The problems associated with performing studies using conventional methods or in the manner described above could make the use of such technology cost-prohibitive. This leads to many treatments failing to reach the market due to lack of funding or to poorly conducted experiments by manufacturers of consumer-grade devices, and unsubstantiated claims that ultimately cost these businesses wasted time and money. There is a pressing need for democratising the studies' procedures and making the medical grade standards available across a range of different applications including in consumer-grade devices.

The present invention addresses the preceding problems of conventional experimental or investigative studies, through a cloud-connected device. The functionality of such a device is illustrated by an example of a heretofore unexplored in this context application of the non-invasive brain stimulation. The invention encompasses conducting major, even all, aspects of an experimental or investigative study on-line, including direct monitoring or evaluation of trial participants.

### SUMMARY

The present disclosure seeks to provide an intervention system that automatically performs unbiased testing of at least one subject under investigative or experimental study.

Moreover, the present disclosure seeks to describe a method for using the intervention system that automatically performs unbiased testing of at least one subject under investigative or experimental study.

An objective of the present disclosure is to provide a solution that overcomes at least partially the problems encountered in existing protocols and systems in the prior art and provides an improved intervention system for investigative or experimental study.

In a first aspect, embodiments of the present disclosure provide an intervention system that, when in operation, automatically performs unbiased testing of at least one subject under investigative or experimental study, wherein the system comprises:
(i) an input device for collecting bibliographic and physiological information from the at least one of subject;
(ii) a memory module for storing the collected bibliographic and physiological information;
(iii) a stimulation arrangement for providing stimulation associated with at least two different types of interventions to the at least one subject; and
(iv) a data processing arrangement configured to be in communication with the stimulation arrangement and the input device to:
   (a) apply at least one randomization algorithms to generate randomization files for one or more subjects, wherein the at least one randomization files comprise a set of files including associated information between the bibliographic information and the types of interventions generated using the at least one randomization algorithm;
   (b) operate the stimulation arrangement to apply at least one intervention from the at least two different types of interventions to the one or more subjects, in accordance with the randomization files generated by the data processing arrangement.

Embodiments of the disclosure are advantageous in terms of providing an intervention system, which has the potential of ensuring blinding of subjects under investigative or experimental study, automating the blinding procedure, making the investigative or experimental study in-house for the subjects, efficient controlling of the equipment during investigative or experimental study and remote and unbiased collection of the data. Furthermore, the system of the present disclosure provides a solution for achieving safe and effective investigative or experimental studies using stimulation(s), and also real-time monitoring of the studies in accordance with the response received from the brain. Moreover, where necessary, the system enables a study expert to conduct the study remotely with efficient controlling of the equipment during investigative or experimental study.

In a second aspect, embodiments of the present disclosure provide a method for using an intervention system that, when in operation, automatically performs unbiased testing of at least one subject under investigative or experimental study, wherein the method includes:
i) using an input device to collect bibliographic and physiological information from the at least one subject;
(ii) storing the collected bibliographic and physiological information in a memory module;
(iii) using a stimulation arrangement to provide stimulation associated with at least two different types of interventions to the at least one subject; and
(iv) using a data processing arrangement that is in communication with the stimulation arrangement and the input device to:
   (a) apply at least one randomization algorithms to generate randomization files for one or more subjects, wherein the at least one randomization files comprise a set of files including associated information between the bibliographic information and the types of interventions generated using the at least one randomization algorithm;
   (b) operate the stimulation arrangement to apply at least one intervention from the at least two different types of interventions to the one or more subjects, in accordance with the randomization files generated by the data processing arrangement.

In a third aspect, embodiments of the present disclosure provide a computer programme product comprising a non-transitory computer-readable storage medium having computer-readable instructions stored thereon, the computer-readable instructions being executable by a computerised device comprising processing hardware to execute the aforementioned method.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary embodiments of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a block diagram of an intervention system for automatically performing unbiased testing of at least one subject under investigative or experimental study, in accordance with an embodiment of the present disclosure;

FIG. 2 is a schematic illustration of a block diagram of a stimulation arrangement providing brain stimulations to the at least one subject, in accordance with an embodiment of the present disclosure; and

FIG. 3 is an illustration of steps of a method for automatically performing unbiased testing of at least one subject, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In a first aspect, embodiments of the present disclosure provide an intervention system that, when in operation, automatically performs unbiased testing of at least one subject under investigative or experimental study, wherein the system comprises:
(i) an input device for collecting bibliographic and physiological information from the at least one of subject;
(ii) a memory module for storing the collected bibliographic and physiological information;
(iii) a stimulation arrangement for providing stimulation associated with at least two different types of interventions to the at least one subject; and
(iv) a data processing arrangement configured to be in communication with the stimulation arrangement and the input device to:
   (a) apply at least one randomization algorithms to generate randomization files for one or more subjects, wherein the at least one randomization files comprise a set of files including associated information between the bibliographic information and the types of interventions generated using the at least one randomization algorithm;
   (b) operate the stimulation arrangement to apply at least one intervention from the at least two different types of interventions to the one or more subjects, in accordance with the randomization files generated by the data processing arrangement.

In another aspect, embodiments of the present disclosure provide a method for using an intervention system that, when in operation, automatically performs unbiased testing of at least one subject under investigative or experimental study, wherein the method includes:
(i) using an input device to collect bibliographic and physiological information from the at least one subject;
(ii) storing the collected bibliographic and physiological information in a memory module;
(iii) using a stimulation arrangement to provide stimulation associated with at least two different types of interventions to the at least one subject; and
(iv) using a data processing arrangement that is in communication with the stimulation arrangement and the input device to:
   (a) apply at least one randomization algorithms to generate randomization files for one or more subjects, wherein the at least one randomization files comprise a set of files including associated information between the bibliographic information and the types of interventions generated using the at least one randomization algorithm;
   (b) operate the stimulation arrangement to apply at least one intervention from the at least two different types of interventions to the one or more subjects, in accordance with the randomization files generated by the data processing arrangement.

The present disclosure provides the aforementioned apparatus and the aforementioned method for performing unbiased testing of at least one subject under investigative or experimental study, when in operation. The system disclosed herein is simple, robust, inexpensive, and allows in-house investigative or experimental study in an efficient manner. The system efficiently ensures blinding of subjects under investigative or experimental study, by applying randomization algorithms and generating randomization files with associated information in a manner that is robust, effective, and adaptive.

Throughout the present disclosure, the terms “patient”, “subject” or “subjects” as used herein relate to any person (i.e., human being) going through an ongoing investigative or experimental study(ies). Optionally, the subject may be a person having a certain physical or mental disorder such as epilepsy, a head injury, encephalitis, brain tumour, encephalopathy, memory related problems, sleep disorders, stroke, dementia etc. Alternatively, the subject may be a person willing to participate in investigative or experimental study for some specific disease, or for a non-clinical application including but not limited to investigative or experimental studies of mental and/or physical performance, of mental well-being and of habits and day-to-day activities. In specific embodiments mentioned below that do not require more than one individual, the subject may be a person willing to conduct his own experiments not participating in any mass investigative or experimental study.

Throughout the present disclosure the term “bibliographic information” refers to factual personal information about the subject including, but not limited to gender, age, occupation, geographical location, name and surname, e-mail address, device ID, app ID and also personal sensitive information including any underlying health conditions, regularly taken medications, any administered surgical interventions, implanted devices, habits that might influence the outcome of the intervention i.e. regular smoking, recreational drug use.

Throughout the present disclosure the term “physiological information” refers to information about the current physiological and psychological state of the patient. The physiological information can be collected from the patients through means of electrophysiological techniques such as electroencephalography (EEG), electrocardiogra (ECG), electromyography (EMG), functional magnetic resonance imaging (fMRI), functional near-infrared spectroscopy (fNIRS), eye tracking, Magnetoencephalography (MEG), Electromyography (EMG), electric field encephalography (EFEG) and also through means of techniques used in psychology such as tests and questionnaires including but not limited to Cognitive Performance tests, Oxford Happiness Inventory, Life Orientation Test, Zung self-rated depression scale.

Throughout the present disclosure, the term “input device” as used herein relates to a device or an arrangement configured to receive bibliographical as well physiological information from the at least one subject via a user interface, wherein the user interface is configured to receive the bibliographical information through at least one of a button interface, a wireless interface, a touch-screen interface, a gesture interface, a microphone interface (voice detection). The user interface is also capable of receiving physiological information through another device capable of measuring such physiological information.

The input device, when in operation, is communicably coupled to a memory module and a data processing arrangement via a communication module, wherein the communication is realised using wired or wireless connections including, but not limited to a connection via the Internet, Bluetooth® and so forth.

Optionally, the input device includes at least one of a smartphone, a computer (can be personal, cloud-based, distributed or a tablet computer), a smart-watch, a remote control, a medical device, and so forth.

Beneficially, the input devices provide a better interaction with the subject through a user-friendly interface. Additionally, the input devices enable the subject to answer the questions asked by the intervention system, in real time manner.

Throughout the present disclosure, the term “memory module” as used herein relates to a volatile or persistent medium, such as an electrical circuit, magnetic disk, virtual memory or optical disk, in which a computer and/or a data processing arrangement may store data for any duration. Optionally, the memory module may be a non-volatile mass storage such as physical storage media.

Throughout the present disclosure, the term “stimulation arrangement” as used herein relates to an arrangement for providing at least two different types of any form of intervention, including but not limited to audio stimulation, visual stimulation, electrical or electromagnetic stimulation, request to perform a test or an exercise, and dispensation of substances, for the purposes of experimental or investigative study to the at least one subject.

In an embodiment, the stimulation arrangement provides brain stimulation to the at least one subject, wherein the stimulation arrangement pursuant to the present disclosure comprises a headwear arrangement including a plurality of electrodes. In use, a plurality of electrodes is placed or positioned on the scalp of the subject, in order to establish an electrical contact with neurons in the brain of the subject. Such electrical contact establishes an electrical path to detect electrical signals generated by the neurons and to provide brain stimuli to the neurons and/or other cells present inside the brain of the subject. The plurality of electrodes detects the electrical signals generated inside the brain of the subject by activity of neurons, wherein the detected electrical signals are provided to an input/output arrangement. The plurality of electrodes may optionally be configured as any suitable EEG electrode arrangement known in the art. The plurality of electrodes are hybrid electrodes which can function as both for EEG recording and/or for electrical stimulation, for example, transcranial current stimulation (tCS), transcranial direct current stimulation (tDCS), transcranial alternating current stimulation (tACS), transcranial random noise stimulation (tRNS), transcranial temporal interference stimulation (TI), transcranial temporal summation (TS) and/or any other arbitrary transcranial electric current stimulation protocol generated by the adaptive algorithms (tES). A plurality of magnetic coils can be used instead of electrodes to deliver transcranial static magnetic field stimulation (tSMS), low field magnetic stimulation (LFMS), repetitive transcranial magnetic stimulation (rTMS) and/or any other arbitrary transcranial magnetic stimulation (TMS) protocol generated by the adaptive algorithms. Alternatively, also, a plurality of ultrasound generators can be used for delivery of Focused Ultrasound Stimulation (FUS) protocols also generated by the adaptive algorithms.

Throughout the present disclosure, the term “adaptive learning algorithm” as used herein relates to software-based algorithms that are executable on computing hardware and are operable to adapt and adjust their operating parameters depending upon information that is presented to while trying to minimize a predefined error/loss metric, or processed by, the software-based algorithms when executed on the computing hardware.

Throughout the present disclosure, the terms “headwear” or “headwear arrangement” as used herein relate to an element of clothing which is worn by the subject on his/her head. Optionally, the headwear arrangement may include, but not be limited to, any one of a cap, a hat, a helmet, headphones, a headband, glasses or a bonnet. More optionally, the headwear arrangement may be fabricated in a manner such that it comprises a layer of electrically insulating or conductive and grounded material. In an example, the headwear arrangement can be fabricated from one of materials including, but not limited to, wool, cotton, polyester, rubber, lycra, nylon or buckram.

Throughout the present disclosure, the term “brain stimulus” or “brain stimuli” (plural of “stimulus”) as used herein relates to an external electrical current or to a defined sequence or multiple sequences of electric current amplitudes between a pair, several pairs or any combination of the electrodes applied to the scalp of a subject or to locations spatially remote from the scalp of a subject, in order to modify and/or enhance an electrical activity in the brain of the subject or in the nervous tissues that the current is able to reach.

In an embodiment, an intensity of intervention provided from the stimulation arrangement may be controlled by the person using the system Moreover, in an example, brain stimuli applied to the scalp of the subject are analogue external electrical signals having a voltage in a range of 1 millivolt to 50 volts and having a current in a range of 0.1 milliampere to 20 milliamperes.

Throughout the present disclosure, the term “data processing arrangement” as used herein relates to programmable and/or non-programmable components that, when in operation, execute one or more software applications for storing, processing and/or sharing of data and/or a set of instructions. Optionally, the data processing unit can include, for example, a component included within an electronic communications network. Furthermore, the data processing arrangement may include hardware, software, firmware or a combination of these, suitable for storing and processing various information and services accessed by the one or more subjects using the one or more subjects' equipment. Optionally, the data processing arrangement may include functional components, for example, a processor, a memory, a network adapter and so forth. For example, the data processing arrangement can be implemented using a computer, a phone (for example, a smartphone), a local server, a server arrangement (such as, an arrangement of two or more servers communicably coupled with each other), a cloud server, a quantum computer and so forth. The Server can be implemented on open-source or commercial OS Unix-like Linux family (Debian, Ubuntu, CentOS, Fedora, SUSE Linux, Red Hat Enterprise Linux) and BSD (NetBSD, FreeBSD, OpenBSD), Microsoft Windows Server, Oracle Solaris and other.

Pursuant to the present disclosure, the input device, the memory module, the stimulation arrangement, and the data processing arrangement are communicably coupled via the communication module. In an embodiment, the data processing arrangement is configured to be in communication with the stimulation arrangement. Furthermore, the input device and the stimulation arrangement are also communicably coupled via a communication interface.

In an embodiment, the input device comprises a smartphone or a tablet with internet connectivity including but not limited to 3G, 4G, 5G, LTE and WiFi and Bluetooth® connectivity including but not limited Bluetooth® Classic, and Bluetooth Low Energy. The smartphone or tablet uses the internet to communicate with the memory module and the data processing arrangement, while using Bluetooth® or internet connection to communicate with the stimulation arrangement and the part of the input device collecting physiological information, thereby acting as a communication module. The input device allows the user to provide inputs for stimulation to be applied to one or more subjects. In an embodiment, the input device communicates with the data processing arrangement to provide stimulation to the user via the stimulation arrangement.

Throughout the present disclosure, the term “randomization” as used herein relates to a process of assigning a subset of subjects for one of the at least two different types of intervention. The term “randomization algorithm” relates to the at least one algorithm used for the process of “randomization” and may include but not limited to pseudo-random, quasi-random and shuffling/permutation algorithms such as linear congruential generator, middle square Weyl sequence algorithm, lava lamp method, quantum noise method, assignment in pseudo-random order, Fisher-Yates shuffling.

The randomization algorithm and how it is applied varies between the different types of study designs, which include but are not limited to parallel trial, cross over trial, response adaptive feedback, ranking and selection feedback, factorial design, and/or randomized withdrawal design. There are also different types of randomization algorithms, which include but are not limited to stratified randomization, blocking, cluster and the combinations of these. Optionally and applicable to all the types of study designs, the at least one subject can be divided into even or uneven “blocks” and assigned random types of intervention within these blocks. More optionally, the at least one subject can be “stratified” into subgroups based on their bibliographic information, then split into blocks within the subgroups and assigned random types of intervention within these blocks.

Throughout the present disclosure, the term “randomization files” refers to a database or a set of files containing associated information between the bibliographic information and the type of intervention generated using the at least one randomization algorithm. Optionally, the randomization files also contain associated information between the bibliographic information and the physiological information. The randomization files may be stored using at least one of the following approaches: text file, Microsoft Excel file, Microsoft Access file, NoSQL Document/JSON Database (CouchDB, Couchbase, MongoDB, exist, Berkeley DB XML), Graph Database (Neo4j, OrientDB, AllegroGraph, Blazegraph, InfiniteGraph, FlockDB, Titan), IBM DB2, SQL Database (SQLite, MySQL, Microsoft SQL Server, PostgreSQL, MariaDB, Oracle Database), Redis, Cassandra. Since the generation of randomization files and associated information in one database does not involve doctors, nor other third parties, the system and method described in this disclosure is capable of achieving the same result as from a gold standard double-blinded placebo-controlled trial with negligible costs on top of subject recruitment.

The data processing arrangement, when in operation, applies the at least one randomization algorithms to generate randomization files for a group of subjects, and operates the stimulation arrangement for the group of subjects in accordance with the randomization files. Further, the data processing arrangement, when in operation, associates the collected physiological information with the bibliographic information in the randomization files to generate “associated information”. In an embodiment, the data processing arrangement acts as a central element of the intervention system as disclosed herein the present disclosure. In other words, all the data or signals to be transmitted from input device to the stimulation arrangement or the information or signals to be transmitted from the stimulation arrangement to the input device has to also pass through the data processing arrangement.

In an adaptive trial embodiment, the system performs a first intervention to the at least one subject in accordance with the randomization files and thereafter based on the collected associated information generates a second set of randomization files on the same or a different plurality of subjects and performs a second intervention in accordance with the second set.

Throughout the present disclosure, the term “stimulation” as used herein relates to any form of intervention, including but not limited to audio stimulation, visual stimulation, electrical or electromagnetic stimulation, and dispensation of substances, for the purposes of experimental or investigative study to the at least one subject.

In an embodiment, the term stimulation relates to altering (referring to raising, lowering or otherwise modulating) levels of physiological or specifically nervous activity in the brain or in the tissues spatially remote from the given subject's brain. Notably, the stimulation of the brain of the subject is carried out with help of electrical signals, applied to the scalp of the subject with the help of one or more electrodes. Further, stimulation of the brain is achieved by using any one of minimally invasive brain stimulation or non-invasive brain stimulation methods, or optionally both.

Throughout the present disclosure, the term “input/output arrangement” as used herein relates to programmable and/or non-programmable components that, when in operation, receive, modify, convert, process or generate one or more types of signals. Optionally, the input/output arrangement is implemented as a hardware or a software, or a combination thereof.

In an embodiment, the system further comprises an external stimulation arrangement for providing at least one of: a visual stimulation, audio stimulation and/or a virtual reality stimulation to the subject's brain. Optionally, the external stimulation arrangement communicates with the data processing arrangement directly or via the input device. More optionally, at least one of: a visual stimulation, an audio stimulation and/or a virtual reality stimulation to the subject's brain, provided by the external stimulation arrangement is in synchronisation with the electrical stimuli applied to the brain of the subject.

In an embodiment, the system comprises a monitoring device, in pursuant to the present disclosure the monitoring device, when in operation is communicably coupled with input device, which is in turn coupled with the data processing arrangement, the stimulation arrangement and the memory module and enables a person using the system to monitor at least one of the randomization files, the collected physiological information, the associated information and the analysed physiological information for the at least one subject.

In an embodiment, the system may include a monitoring device having a secured access that restricts user access to prevent user access to prevent unblinding of data of a user to another till the completion of the investigative or experimental study. The monitoring device may be password-protected and may restrict user access to prevent unblinding of the data to some users or any users at all until the trial is complete. Advantageously, the system disclosed in this embodiment executes functions without having to disclose the contents of the randomisation file to any user or subject, thereby excluding any possibility of bias or unblinding through inadvertent disclosure of the contents of the randomization file.

Throughout the present disclosure, the term “user” or “person” or “trial designer” as used herein relates to at least one of a specialist designing the study protocol, a doctor conducting the investigative or experimental study or the nursing staff involved in the study.

In an embodiment, the system comprises a sensing arrangement, in pursuant to the present disclosure the sensing arrangement, when in operation is coupled with the input device and may include at least one sensor to collect at least one parameter required to ensure a correct execution of the experimental or investigative study on the at least one subject. The at least one sensor may include but not limited to ammeter, voltmeter, impedance measurement circuitry, heart rate sensing devices, galvanic skin response sensors, muscle movement sensors, face recognition apps, IR sensors, glucose level sensors, hormone detectors, gait trackers, tremor trackers and questionnaire on the input device.

Throughout the present disclosure, the term “intervention” as used herein relates to delivering the at least one element of the experimental treatment to the subject under the investigative or experimental study. These interventions may include but are not limited to methods that fall under the definition of stimulation and stimulation arrangement, procedures; or changes to subjects' behaviour, such as diet, well-being routines and exercises.

According to an embodiment, the invention as described in the present disclosure involves a stimulation arrangement in communication with an input device. The stimulation arrangement and the input device are connected to a data processing arrangement via a communication module. In other words, the input device and the stimulation arrangement both interact with the user and could be implemented as a single device.

In an embodiment, the input device allows the user to interfere with the algorithms and randomization files and provide inputs for stimulation to one or more subjects. Any such interference, particularly where the user is no longer blinded to the type of intervention as recorded above is recorded in the memory module in a secure and immutable manner.

In an embodiment, one or more stimulation protocols as interventions have been provided at the data processing arrangement. The data processing arrangement includes a randomization protocol to randomly assign the one or more stimulation protocols as interventions. The user wearing the stimulation apparatus may activate the stimulation protocols via the input device. The interventions are applied to the subject via a stimulation arrangement. The input device communicates with the data processing arrangement for random assignment of the one or more stimulation protocols as interventions to the user. The stimulation arrangement stimulates the user on receiving the stimulation interventions from the data processing arrangement via the input device. While stimulating the user via the stimulation arrangement, the input device allows the user to answer one or more questions from questionnaire to check on the user's performance while he is subjected to the stimulation and/or to collect physiological data from other devices. Since there is no involvement of any third party in any interactions with the subject, provided that sufficient prior instruction for use has been given through a manual or a video guide, the present invention advantageously allows the user to operate the intervention system without any external aid. This is advantageous for elimination of human factors, care standards, expectations and biases in such trials. Optionally, the data processing arrangement generates an index indicating a score of the user's performance while being subjected to at least a type of intervention.

Furthermore, in another embodiment, the user may also be subjected to physical and mental performance tests before, during or after the stimulation. The tests allow the user to answer one or more questions from questionnaire or to perform one or more tasks to check on the user's performance and to collect the data from the tasks performed including, but not limited to physiological data, accelerometer data, images and videos. In an embodiment, the data is stored in the memory module and is passed further to the processing arrangement to generate a score. After unblinding of the randomization files the score is subsequently used to assess the efficacy of the one or more types of interventions.

In another embodiment, other than questionnaire, the outcomes of the stimulation result (experimental or investigative interventions) can also be collected through a number of connected devices include EEG/EFEG/MEG, sensing devices, heart rate sensing devices, galvanic skin response sensors, muscle movement sensors, face recognition apps, IR sensors for cancer detection, glucose level sensors, hormone detectors, gait trackers, tremor trackers, and so forth.

In an embodiment, the randomization files may assign two different interventions to the at least one subject undergoing multiple interventions. Specifically, in a cross-over study design the randomization file contains random assignment of at least two different types of intervention to the at least one subject as the first intervention and then swaps the assignment for a subsequent intervention. This embodiment also applies when only one subject is involved in a study and receives at least two different types of interventions in several intervention sessions in random order.

In an embodiment, the intervention is selected from a group of electrical, magnetic, audio, visual, light, ultrasound and binaural beats stimulation.

In an exemplary embodiment, the specialist designs a study protocol which includes the description of the randomization procedure for at least two different types of interventions. The input device collects the bibliographic information about the subject and shares the bibliographic information with the memory module. The data processing arrangement further generates randomization protocol using the outcome stored in the memory module, which results in assignment of control or real intervention to subjects under investigative or experimental study in accordance with the study design and the randomization algorithm. Thus, the assigned protocol (control vs real) is automatically delivered through the stimulation arrangement to the subject without involving a third party to ensure an unbiased blinding of the subjects. Further, during such interventions, the input device allows to collect the data on outcome remotely through a plethora of connected devices tracking various health parameters. In this exemplary embodiment, the outcome data from the at least one subject under investigative or experimental study is communicated to the memory module and the data processing arrangement compares the outcomes of the control treatment and the experimental intervention. Further, the at least one subject goes through the control and the experimental intervention in order defined in randomization files.

In another embodiment, the specialist designs the adaptive trial. This means that the assignment of the participants to the different interventions is influenced by the data collected from the previous participants. If a particular intervention has been shown to be more effective in the previous participants, the probability of the assignment to this condition increases.

The randomization algorithm and how it is applied varies between the different types of study designs, which include but are not limited to parallel trial, cross over trial, response adaptive feedback, ranking and selection feedback, factorial design, randomized withdrawal design. There are also different types of randomization algorithms, which include but are not limited to stratified randomization, blocking, cluster and the combinations of these.

In another embodiment, the stimulation arrangement, the monitoring device and the sensing arrangement are connected to a cloud server (such as the data processing arrangement) and act like a single device.

Beneficially, the randomization algorithm contributes largely to achieving a more cost effective and secure process of unbiased blinding of the plurality of the subjects and the specialist. Additionally, implementation of randomization algorithms helps in enhancing the therapeutic contribution of the stimulation arrangement in the investigative or experimental studies.

In a particular embodiment, the present disclosure discloses that the intervention system as described herein can also be considered an ideal device where there is a requirement of unblinding the data before the completion of a trial. Conventionally, unblinding the data prior to completion of the trial may lead to distorting the whole purpose of clinical trials and also skew the results. This problem is often encountered in several of the clinical trial situations. However, the intervention system as disclosed by the present invention creates a plurality of “chinese walls” between the participant and anyone else in the trial. A Chinese wall can be considered a virtual barrier erected to block the exchange of information between participants and anyone else in the trial. Consequently, with the employment of the disclosed intervention system as an autonomic system and the Chinese walls, there is no danger of skewing the results, even if the data is unblinded before the trial is completed. Therefore, since the disclosed system can be autonomic, there is a potential possibility to unblind the data as the study progresses. The above practice has only been made possible because there is no involvement or requirement of a third party while conducting the clinical trials.

The present disclosure also relates to the method as described above. Various embodiments and variants disclosed above apply mutatis mutandis to the method.

Optionally, the method includes using a monitoring device that having a secured access that, when in operation, restricts user access to prevent unblinding of data of a user to another until the completion of the investigative or experimental study.

Optionally, any data including bibliographical and physiological information as well as stimulation protocol information, that is passed between any of the communicably coupled devices is protected using secure encryption.

Optionally, the randomization files assign two different interventions to the at least one subject undergoing multiple interventions.

Optionally, the method includes using at least one randomization algorithm for generating randomization files.

Optionally, an intensity of stimuli provided from the stimulation arrangement is controlled by the person using the system.

Optionally, the method includes using a sensing arrangement that, when in operation, measures at least one parameter required for ensuring a correct execution of the experimental or investigative study on the at least one subject.

Optionally, the intervention is selected from a group of electrical, magnetic, audio, visual, light, ultrasound and binaural beats stimulation.

In an embodiment, the present disclosure provides a computer programme product comprising a non-transitory computer-readable storage medium having computer-readable instructions stored thereon, the computer-readable instructions being executable by a computerised device comprising processing hardware to execute a method of using a brain interfacing apparatus that provides, when in operation, brain activity monitoring and stimulation of the brain of the subject.

### DETAILED DESCRIPTION OF THE DRAWINGS

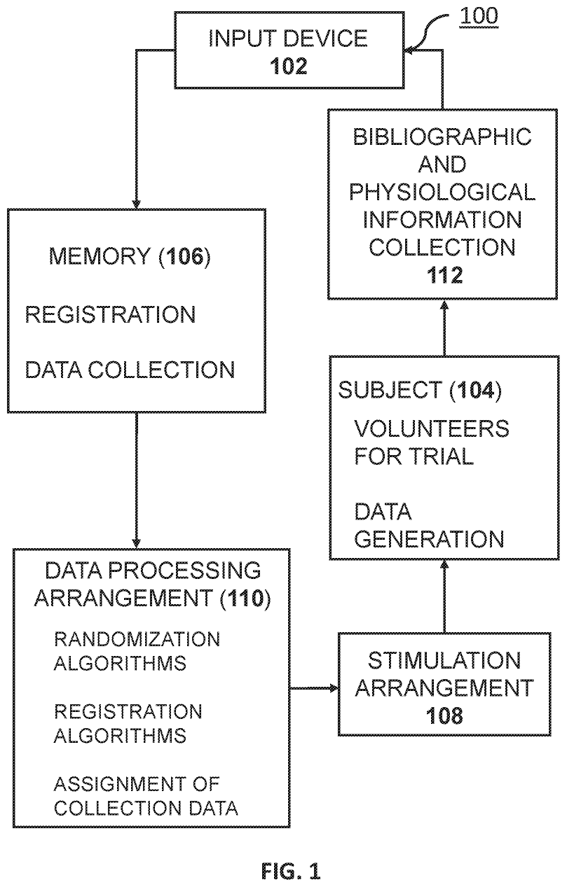

Referring FIG. 1, there is shown a block diagram of an intervention system for automatically performing unbiased testing of at least one subject under investigative or experimental study, in accordance with an embodiment of the present disclosure. The arrows indicate the flow of the steps in the system, starting with the subject 104. As shown, the intervention system 100 comprises an input device 102 for collecting bibliographic and physiological information 112 from the at least one of subject 104, a memory module 106 for storing the collected bibliographic and physiological information, an arrangement 108 for providing at least two different types of interventions to the at least one subject 104 and a data processing arrangement 110. When in operation, the input device 102, memory module 106, the stimulation arrangement 108 and the data processing arrangement 110 are communicably coupled via communication modules (not shown). The data processing arrangement 110 algorithmically generates randomization files for a group of subjects, and stores the randomization files in the memory module 106. Further, the data processing arrangement 110, when in operation, operates the stimulation arrangement 108 via the input arrangement 102 for the group of subjects in accordance with the randomization files stored in the memory module 106. The physiological information 112 received from the subject 104 via the sensors or questionnaires (not shown) as an outcome of the intervention, is then collected by the input device and passed to the memory module and the data processing arrangement thereafter, where it's stored and processed in conjunction with the randomization files.

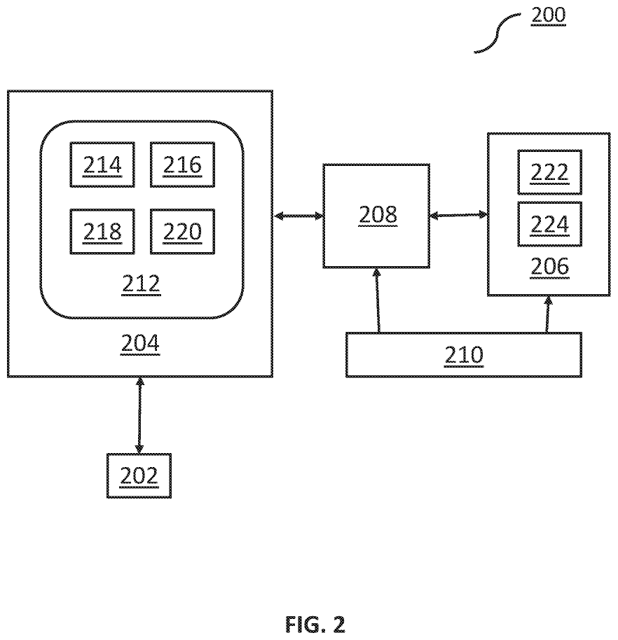

Referring to FIG. 2, there is a stimulation arrangement 200 (such as the stimulation arrangement 108 of FIG. 1) for providing brain stimulations to at least one subject 202 (such as the at least one subject 104 of FIG. 1), in accordance with an embodiment of the present disclosure. As shown, the stimulation arrangement 200 comprises a headwear arrangement 204, a data processing arrangement 206 (such as the data processing arrangement 110 of FIG. 1), an input/output arrangement 208 and one or more power units 210. Furthermore, the headwear arrangement 204 comprises an electrode arrangement 212 including a plurality of electrodes 214 to 220, wherein the plurality of electrodes is arranged in a manner to make contact with the scalp of the user, for detecting the brain activity. Moreover, the electrode arrangement 212 is communicably coupled to the input/output arrangement 208, wherein the input/output arrangement 208, when in operation, receives the detected signals and delivers the brain stimuli to the at least one of the plurality of electrodes 214 to 220. Furthermore, the input/output arrangement 208 contains an optional input signal pre-processing arrangement (not shown), which can include an optional amplifier (not shown); an artefact filter (not shown); an input converter (not shown); an output converter (not shown) and stimuli generator (not shown). Furthermore, the input/output arrangement 208 is communicably coupled with the data processing arrangement 206. Moreover, the data processing arrangement 206 comprises a memory module 222 and a processing unit 224. The one or more power unit 210, when in operation, provides electrical power to the input/output arrangement 208 and the data processing arrangement 206.

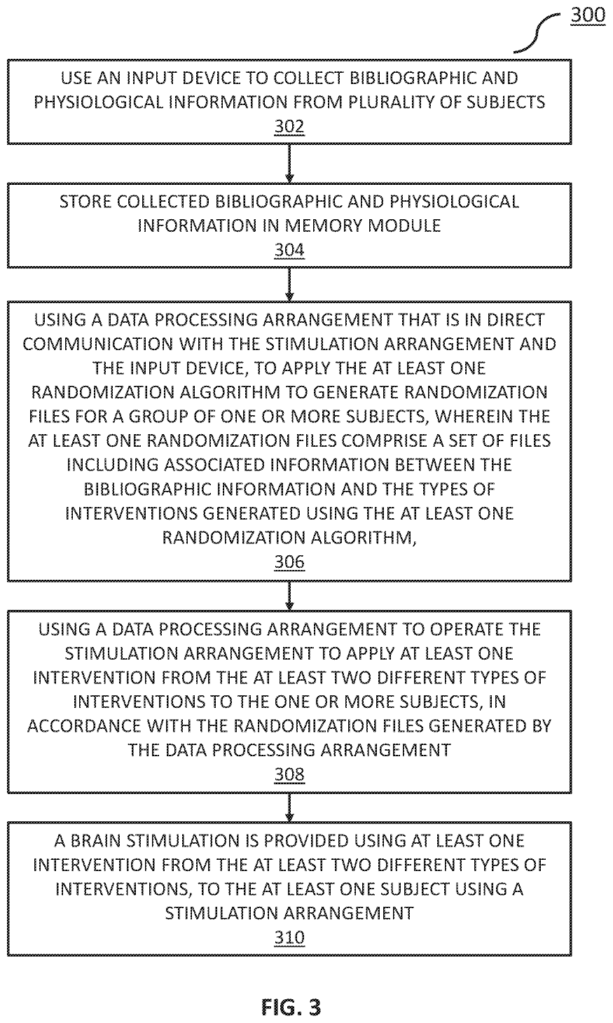

Referring to FIG. 3, illustrated are steps of a method 300 for automatically performing unbiased testing of at least one subject (such as the at least one subjects 104 of FIG. 1) under investigative or experimental study using an intervention system (such as the intervention system 100 of FIG. 1), in accordance with an embodiment of the present disclosure. The method initiates at a step 302. At the step 302, bibliographic information is collected from the at least one subject using an input device (such as the input device 102 of FIG. 1). At a step 304 the collected physiological information is stored in a memory module (such as the memory module 106 of FIG. 1). At a step 306, a data processing arrangement that is in communication with the input device and the stimulation arrangement, is used to apply the at least one randomization algorithm to generate randomization files for the at least one subject, wherein the randomization files comprise a set of files including associated information between the bibliographic information and the types of interventions generated using the at least one randomization algorithm. At a step 308 a data processing arrangement is used to operate the stimulation arrangement to apply at least one intervention from the at least two different types of interventions to the one or more subjects, in accordance with the randomization files generated by the data processing arrangement. At a step 310 a brain stimulation is provided using at least one intervention from the at least two different types of interventions, to the at least one subject using a stimulation arrangement (such as the stimulation arrangement 108 of FIG. 1). At the step 310 the method 300 ends.

The steps 302 to 310 of method 300, are only illustrative and other alternatives can also be provided where one or more steps are added in accordance with the other disclosed embodiments of the invention, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

1. An intervention system that, when in operation, automatically performs unbiased testing of at least one subject under investigative or experimental study, wherein the system comprises:
(i) an input device for collecting bibliographic and physiological information from the at least one of subject;
(ii) a memory module for storing the collected bibliographic and physiological information;
(iii) a stimulation arrangement for providing stimulation associated with at least two different types of interventions to the at least one subject; and
(iv) a data processing arrangement configured to be in communication with the stimulation arrangement and the input device to:
(a) apply at least one randomization algorithms to generate randomization files for one or more subjects, wherein the at least one randomization files comprise a set of files including associated information between the bibliographic information and the types of interventions generated using the at least one randomization algorithm;
(b) operate the stimulation arrangement to apply at least one intervention from the at least two different types of interventions to the one or more subjects, in accordance with the randomization files generated by the data processing arrangement.

2. An intervention system of claim 1, wherein the system comprises a monitoring device having a secured access that, when in operation, restricts user access to prevent unblinding of data of a user to another until completion of the investigative or experimental study.

3. An intervention system of claim 1, wherein the randomization files assign two different interventions to the at least one subject undergoing multiple interventions.

4. An intervention system of claim 1, wherein the data processing arrangement further analyses the physiological information collected from the at least one subject, before and after the intervention.

5. An intervention system of claim 1, wherein the data processing arrangement performs a second intervention to the at least one subject, based on the analysed physiological information.

6. An intervention system of claim 1, wherein an intensity of intervention provided from the stimulation arrangement is controlled by the person using the system.

7. An intervention system of claim 1, wherein the system comprises a sensing arrangement that, when in operation, measures at least one parameter required to ensure a correct execution of the investigative or experimental study on the at least one subject.

8. An intervention system of claim 1, wherein the intervention is selected from a group of electrical, magnetic, audio, visual, light, ultrasound and binaural beats stimulation or any combination thereof.

9. A method for using an intervention system that, when in operation, automatically performs unbiased testing of at least one subject under investigative or experimental study, wherein the method includes:
(i) using an input device to collect bibliographic and physiological information from the at least one subject;
(ii) storing the collected bibliographic and physiological information in a memory module; (iii) using a stimulation arrangement to provide stimulation associated with at least two different types of interventions to the at least one subject; and
(iv) using a data processing arrangement that is in communication with the stimulation arrangement and the input device to:
(a) apply at least one randomization algorithms to generate randomization files for one or more subjects, wherein the at least one randomization files comprise a set of files including associated information between the bibliographic information and the types of interventions generated using the at least one randomization algorithm;
(b) operate the stimulation arrangement to apply at least one intervention from the at least two different types of interventions to the one or more subjects, in accordance with the randomization files generated by the data processing arrangement.

10. A method of claim 9, wherein the method includes using a monitoring device having a secured access that, when in operation, prevents unblinding of data of a user to another until completion of the investigative or experimental study.

11. A method of claim 9, wherein the randomization files assign two different interventions to the at least one subject undergoing multiple interventions.

12. A method of claim 9, wherein the method includes using the data processing arrangement for analysing the physiological information collected from the at least one subject, before and after the first intervention.

13. A method of claim 9, wherein the method includes using the data processing arrangement for performing a second intervention to the at least one subject, based on the analysed physiological information.

14. A method of claim 9, wherein an intensity of intervention provided from the stimulation arrangement is controlled by the person using the system.

15. A method of claim 9, wherein the method includes using a sensing arrangement that, when in operation, measures at least one parameter required for ensuring a correct execution of the investigative or experimental study on the at least one subject.

16. A method of claim 9, wherein the intervention is selected from a group of electrical, magnetic, audio, visual, light, ultrasound and binaural beats stimulation or any combination thereof.

17. A computer programme product comprising a non-transitory computer-readable storage medium having computer-readable instructions stored thereon, the computer-readable instructions being executable by a computerised device comprising processing hardware to execute a method of claim 9.
